(19) United States
(12) Patent Application Publication
(10) Pub. No.: US 2005/0113883 A1
(43) Pub. Date: May 26, 2005

(54) Electromyogram-triggered neuromuscular stimulation device and method

(76) Inventors: Thomas Sandgaard, Littleton, CO (US); Alexander Russo, Littleton, CO (US)

Correspondence Address: THE MCCALLUM LAW FIRM, LLC, 132 KOLAR COURT, ERIE, CO, 80516, US

(21) Appl. No.: 10/772,853

(22) Filed: Feb. 5, 2004

Related U.S. Application Data
(60) Provisional application No. 60/523,635, filed on Nov. 20, 2003.

(51) Int. Cl. A61N 1/18

(52) U.S. Cl. 607/48

(57) ABSTRACT

The methods disclosed herein are of use for the treatment of muscular deficiencies. A Pavlovian system of reward is used to reinforce the efforts of an individual to contract an impaired muscle. The device detects patterns of electrical impulses sent from the brain to an impaired muscle and measures the strength of the impulses against a threshold value. If an impulse exceeding the threshold value is observed, current is sent to electrodes causing the muscle to contract. If an attempt is registered that does not meet the threshold value, the threshold value is lowered and the individual makes another attempt. This novel muscular therapy could potentially rehabilitate thousands of stroke and spinal cord injury victims each year.

### BACKGROUND OF THE INVENTION

1. Field of the Invention

The present invention relates to the neuromuscular rehabilitation of an individual suffering from impaired muscular function; and, specifically to a rehabilitation method effective for stroke and spinal cord injury patients in order to develop functional use of an impaired or paralyzed muscle.

2. Description of Related Art

Each year millions of people worldwide experience neurological disorders such as stroke, spinal cord injury, traumatic brain injury, cerebral palsy and drop foot syndrome. All of these disorders can include impaired muscle function. Currently, there are more than 6 million stroke survivors in the United States alone. Of all stroke survivors, 70% experience decreased mobility in the form of decreased balance and coordination, muscle spasticity, and/or paralysis, mainly in the form of hemiplegia (paralysis of one side of the body). In addition, approximately 11,000 spinal cord injuries occur each year from automobile, sport and industrial related accidents. Overall, it is estimated that 250,000-400,000 people in the United States are living with spinal cord injuries.

Traditional physical therapy is usually prescribed to patients experiencing muscular deficiencies. These therapy sessions must be supervised by licensed therapists, are expensive, and require individuals to travel to a designated care facility. Traditional physical therapy may also be accompanied by electrostimulation or biofeedback techniques. Electrostimulation contracts a paralyzed muscle using external stimuli. This technique, while capable of reducing muscle atrophy, does not encourage the brain to find neurological pathways capable of “firing” the muscle. Biofeedback, or electromyography (EMG), is a technique that utilizes the measurement of a patient's input; however, no assistance in muscle contraction is provided.

Biofeedback equipment currently available today filters and averages measured signals in order to provide slow and easy-to-read data. Thus, a large and steady signal is required to register that an attempt to operate the muscle has been made and requires large and steady changes in the patient's contraction of a muscle, before the change is actually reflected in the read-out. Currently, no easy-to-use, therapeutic option is capable of detecting small changes in electrical impulses sent from the brain to the paralyzed muscle in order to facilitate a method of re-education of damaged communication pathways in order to restore muscle function.

### DETAILED DESCRIPTION

In accordance with the purpose(s) of this invention, as embodied and broadly described herein, this invention, in one aspect, relates to the use of a device for teaching a person suffering from muscle impairment or paralysis to utilize new neurological pathways in the brain to stimulate the affected muscle(s).

The present invention may be understood more readily by reference to the following detailed description of particular embodiments of the invention included therein.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present devices and/or methods are disclosed and described, it is to be understood that this invention is not limited to specific detection or manufacturing techniques, as such may, of course, vary, unless it is otherwise indicated. It is also to be understood that the terminology used herein is for the purpose of describing particular embodiments only and is not intended to be limiting.

### FIGURES

The following drawings form part of the present specification and are included to further demonstrate certain embodiments. These embodiments may be better understood by reference to one or more of these drawings in combination with the detailed description of specific embodiments presented herein.

FIG. 1 is a schematic illustrating the components of a particular embodiment of the present invention.

FIG. 2 illustrates the algorithm utilized with a particular embodiment of the present invention to produce an EMG level display.

FIG. 3 illustrates the algorithm utilized to effect the automatic threshold adjustment in a therapeutic method of the present invention.

### DEFINITIONS

For the purposes of the present invention, the following terms shall have the following meanings:

Moreover, for the purposes of the present invention, the term “a” or “an” entity refers to one or more of that entity; for example, “a muscle” or “an electrical impulse” refers to one or more of those elements or at least one element. As such, the terms “a” or “an”, “one or more” and “at least one” can be used interchangeably herein. It is also to be noted that the terms “comprising,” “including,” and “having” can be used interchangeably. Furthermore, an electrical stimulation “selected from the group consisting of” refers to one or more of the elements in the list that follows, including mixtures (i.e. combinations) of two or more of the elements.

For the purposes of the present invention, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

For the purposes of the present invention, the terms “electrical impulse” and “signal” are interchangeable. Additionally, a “neurological pathway” is a series of synapses within an individual that includes a communication network from the brain to a muscle that allows an electrical signal to travel from the brain to a muscle and back again.

For the purposes of the present invention, “an attempt” is an effort on the part of the patient to contract an impaired muscle. An attempt is usually detected by a detection methodology capable of picking up an electrical signal/impulse at the location of the muscle.

For the purposes of the present invention, the terms “impaired muscle”, “affected muscle”, “motor deficiency”, “muscular insufficiency” or “muscular deficiency” may describe a partially functional muscle or a completely paralyzed muscle.

For the purposes of the present invention, the term “sensory” shall refer to any stimulus affecting the senses of a subject. Exemplary stimuli include, but are not limited to, auditory, visual, physical, olfactory, vibrational, and/or light.

Finally, for the purposes of the present invention, the terms “individual”, “patient”, “victim”, “subject” and “survivor” are to mean a mammal of either gender suffering from a muscular deficiency. In a particular embodiment, the mammal is a human.

Reference will now be made in detail to particular embodiments of the invention.

The present invention embodies devices and methods related to a novel method of muscular therapy. The methods are useful for treating patients with impaired muscular function as they can result in the development of new neurological pathways to stimulate the affected muscle(s). In one embodiment, the present invention provides for methods of recreating a communication network between an individual's brain and his or her impaired muscle. This could result in restored muscular function in such treated individual.

In another embodiment the methods of the present invention could be utilized to detect electrical impulses that are too weak to contract a muscle. The detection of such a weak signal would cause the device to send an electrical impulse to an electrode attached to the skin adjacent the affected muscle, thereby causing the muscle to contract. The sensitivity of the methods of the present invention is an improvement over those currently known in the art. The sensitivity is more than ten times greater than that found in competing products on the market. The improved sensitivity is provided by methods of the present invention that suppress noise from external sources, as well as internal noise from within the patient's body. Additionally, the microprocessor is sampling the input signal at a higher rate than previous methods and using the root-mean-square (rms) value at each particular sampling time point to process and display. The increased sampling rate results in identification of actual attempts and a decrease in the number of “missed” attempts that may fall between the sampling time points with previous methods. The increased sensitivity and sampling time points allow for positive feedback in individuals with very low electrical impulses due to injury. The overall result is that very few attempts to contract a muscle are “missed”, even when no observable movement has been made by the patient, and the efforts of the patient are reinforced in a consistent and motivating way.

According to particular embodiments of the methods of the present invention, a threshold limit is determined such that an impulse from the brain must be of a predetermined strength to register as an attempt to move a targeted muscle. This threshold limit is set as a goal for the patient. When the patient produces an electrical impulse that meets the threshold limit, the muscle is contracted by an electrical signal sent from the device to the electrodes attached to the skin and the threshold is automatically reset to a higher limit. However, when the patient does not meet the threshold for causing a contraction, the threshold is lowered. In this way, positive attempts are reinforced by the reward of a physically definable muscle contraction and insufficient attempts are not penalized.

In another embodiment of the present invention, the threshold is preset at 48 dB when the device is turned on. Every 15 seconds, the threshold is adjusted to a lower setting if attempts during that 15 second period measured 4 dB or more below the original threshold. The new, lower threshold is set 4 dB above the highest EMG reading observed in the 15 second period. If the patient succeeds in reaching the threshold, electrical stimulation produces a muscle contraction and the abbreviation “Stim” is shown on the display along with the output in milliamps. The automatic adjustment of the threshold in a logical and predictable manner works to teach the patient how to regain control in an impaired or paralyzed muscle.

According to another particular embodiment of the present invention, following the stimulation period, a rest period is initiated by a flashing instruction and/or voice command to “Relax” or “Rest”. The rest period trains the patient to relax the muscle, which is an important component of proper muscular function and is just as important as muscular contraction as it functions in overall muscle control. This relaxation period allows the charge on the electrodes and skin to dissipate so that false readings do not occur. Following the relaxation period, the threshold is reset 3 dB above the highest EMG level recorded and the verbal and/or visual command “Ready” is given.

According to a particular embodiment of the present invention, a current column on the display moves vertically three times before moving horizontally to the left, at which point it becomes part of a historical data set. The vertical movement of the current column produces results with a “real-time” feel.

In another embodiment of the present invention, a sensory signal in the form of an audio signal is used to instruct and encourage the patient.

In another embodiment of the present invention, the device screen/display is tilted so that an individual in a reclining position can easily view it. This allows the user to focus on the therapy session instead of balancing the display on his or her lap. The display is updated at least every ⅙ second. Additionally, in a particular embodiment, the display may show 60 seconds of continuous history as a motivational tool to help the patient attempt to contract the muscle or relax. A historical display, which, for example, may show 60 seconds of data, can allow the patient to compare a current attempt with one several seconds in the past.

According to a particular embodiment of the present invention, large electrodes may be used to facilitate ease of placement by an individual with motor deficiencies. The electrodes are capable of being switched, using a relay, from being inputs for EMG monitoring, to being outputs for an externally produced, neuromuscular electrical stimulation (NMES) signal transmitted to the patient. The large size of the electrodes is conducive to nonspecific placement. In a particular embodiment, the electrodes range in size from 1 to 4 inches. In another particular embodiment, the electrode may cover approximately 2 square inches of skin. If a human patient is administering the electrodes to him or herself, approximate placement will suffice. If muscular insufficiencies include the hands, this allows for inexact placement by the patient, rather than necessitating placement by another person.

In another embodiment of the present invention, the movement of a muscle near the targeted muscle does not trigger a positive response. Additionally, movement by other parts of the patient's body does not trigger a positive response. Such movement may be movement of other body parts; such as the opposing limb or the head; as well as general spastic movement of various muscles throughout the patient's body.

In another embodiment of the present invention, the patient has received an injury to the spinal cord. In a particular embodiment, the method of the present invention while in the spinal-cord injury mode is 5 times more sensitive than while in the stroke rehabilitation mode.

According to a particular embodiment of the present invention, the method claimed herein can store a record of one or more therapy sessions for later review by a doctor or other health professional.

According to another particular embodiment of the present invention, the EMG sensitivity is about 1.25-1,000 μV in stroke rehabilitation mode. According to an alternate embodiment of the present invention, the EMG sensitivity is about 0.25 μV to 25 μV or 0.50 μV to 50 μV in spinal cord injury mode.

In another particular embodiment of the present invention, the time interval between the onset of the injury or condition causing the muscular insufficiency and treatment with the methods of the present invention does not impact the effectiveness or long-term success of the therapy.

In another particular embodiment, the improvements are maintained long-term. The improvements may be in the form of improved strength, range-of-motion, tone, flexibility, motor capability or reduced spasticity, for example.

In another embodiment of the present invention a patient may use the methods without the supervision of a licensed therapist.

In another embodiment of the present invention a floating, amplified ground acts to reduce electrical noise disturbance.

According to a particular embodiment of the present invention, a wire may be either real or virtual meaning that an electrical signal may be sent through a conductive length of metal or an electrical signal may be converted into another form of transferable data, for example an infrared signal, and transferred to a microprocessor without the need for direct contact between the sensor and microprocessor.

In another embodiment of the present invention, the methods could be used for research purposes.

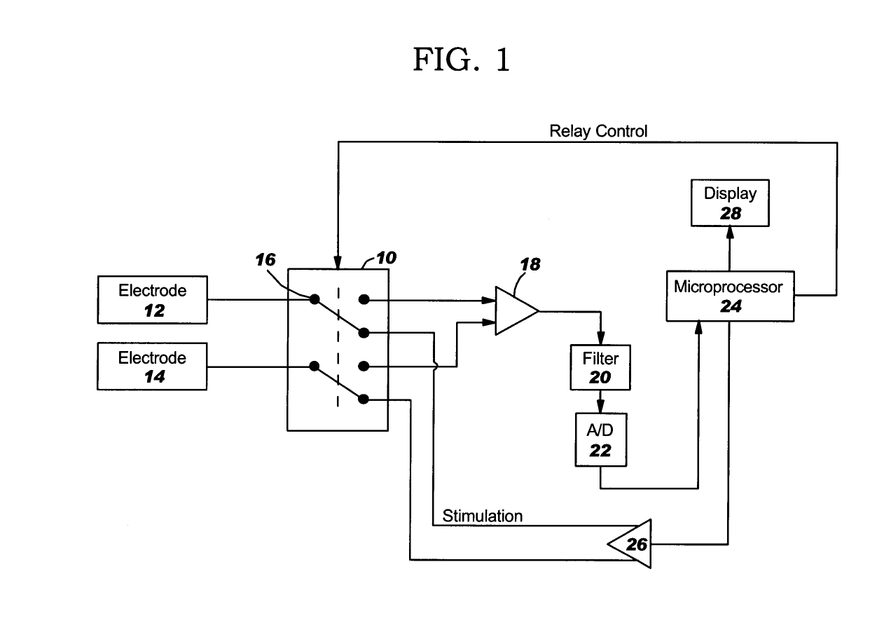

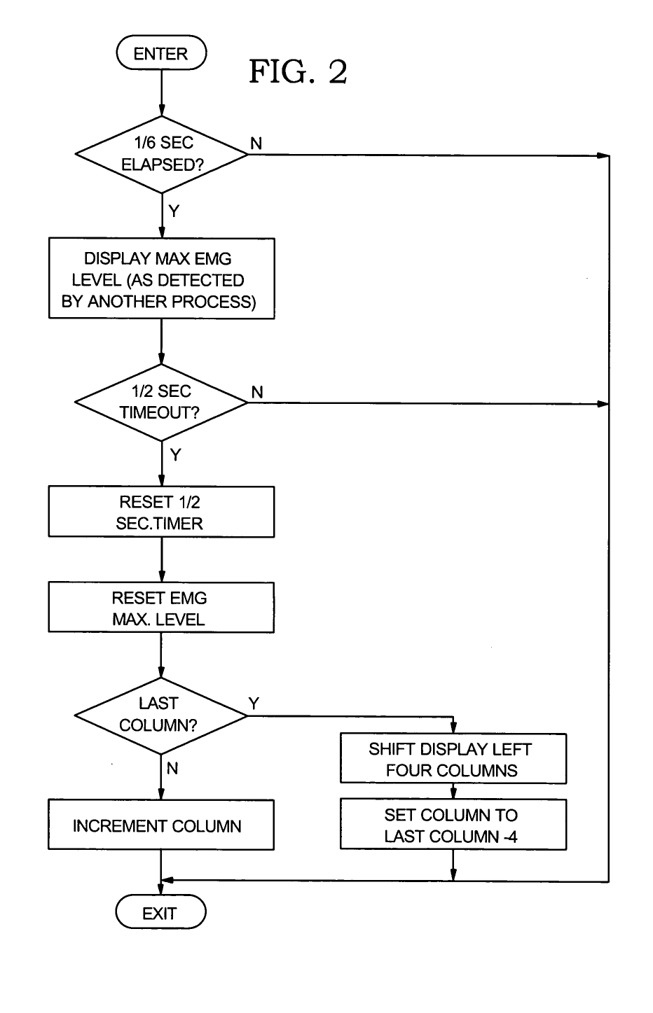

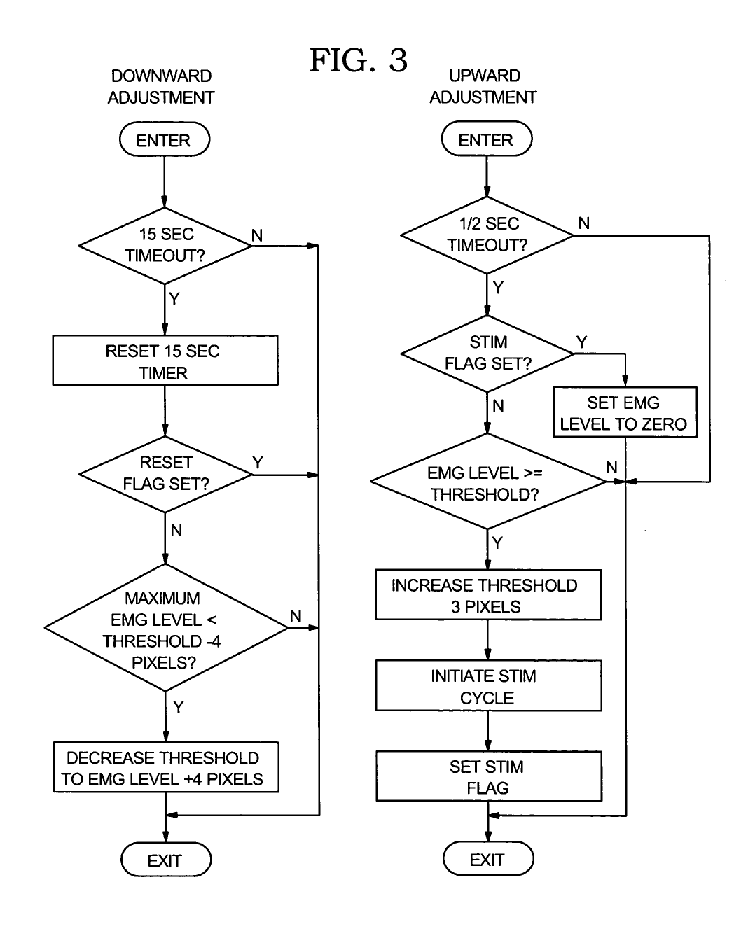

FIG. 1 diagrams the components in a particular embodiment of the present invention. In this particular embodiment, two electrodes 12 and 14 are attached to the skin of the patient at the location of the impaired muscle. The electrodes are then wired to a monitor/stimulation device 10, which houses a relay 16 for switching between the monitoring and stimulation modes. In the monitoring mode, signals from the electrodes 12 and 14 pass through the relay 16 to an amplifier 18 and a filter 20. The signal from the filter 20 is converted into digital form by an analog to digital converter 22, and is applied to a microprocessor 24. The microprocessor 24 is programmed with an algorithm to recognize whether the brain-generated impulse exceeds a threshold, which is set in accordance with the algorithms shown in FIGS. 2 and 3. Upon recognizing a brain-generated impulse which exceeds this pre-set threshold, the microprocessor 24 switches the relay 16 and activates an amplifier/signal generator 26 to generate and supply the electrical stimulation signal to the electrodes 12 and 14, during the stimulation phase of operation. The signal from filter 20 is rectified, to allow for the detection of negative signals, and is compressed by a logarithmic amplifier 26. The electrical stimulation signal generated by the amplifier/signal generator 26 causes the muscle to contract. Information received by and sent from the microprocessor 24 is visible on a display 28 and functions as a positive-feedback mechanism to the patient using the device.

FIG. 2 illustrates the algorithm utilized with a particular embodiment of the present invention to produce the EMG level display. When the device is turned on, operation is set to EMG monitoring (“Ready”) mode, a ½ second display timer is started, and the maximum EMG level reading is cleared. A query is then sent to determine if 1/6th second of the ½ second has elapsed. If the answer to the query is “yes”, the maximum EMG level detected during the ½ second interval is displayed in the current display column. If the answer to the query is “no”, the routine is exited. A query is then issued to determine how the display timer is progressing. If the total elapsed time for the interval has been ½ second, the timer and the maximum EMG level are reset. Another query is then issued to determine if the level displayed is at the last (far right) column of the display. If it is in that position, the display is shifted four columns to the left and the new column is set to the last column minus four. If the answer to the query is “no”, the display column is then incremented.

In summary, while in the EMG level monitoring (“Ready”) mode, display columns are redrawn every ½ second, ad infinitum. During that ½ second, the column information is updated every 1/6th second or three times before it is set. The result of continually querying is to produce accurate, real-time results on the display.

FIG. 3 illustrates the algorithms utilized to effect the automatic threshold adjustment in a therapeutic method of the present invention. When the device is turned on, it is initially set in the EMG monitoring (“Ready”) mode, a 15 second threshold timer is started, and a ½ second display timer is started.

The downward adjustment algorithm is entered and a query is then issued to determine if 15 seconds has elapsed. If it has, the timer is reset and the Rest mode flag is queried. If Rest mode has been detected, the routine is exited. This prevents the threshold from being changed while in Rest mode. If Rest mode has not been detected, another query is issued to determine if the maximum EMG level detected during the last 15 second interval is less than the threshold by four pixels or more. If the answer to the query is “yes”, a new threshold limit is set to the maximum EMG level detected plus four pixels. If the answer is “no”, the program is exited without adjusting the threshold. The threshold is checked for downward adjustment every 15 seconds, ad infinitum.

FIG. 3 also illustrates the algorithm utilized to effect an upward adjustment of the threshold. The upward adjustment algorithm is entered and a query is issued to determine if ½ second has elapsed on the timer. Note: this is the same ½ second timer used in the EMG level display algorithm. If the time has elapsed, the Stimulation flag is set. If the Stimulation mode is detected, the EMG level is set to zero and the routine is exited. This prevents EMG level columns from being displayed during Stimulation mode. If Stimulation is not in progress, another query is issued to determine if the current EMG level is greater than or equal to the threshold level. If it is, the threshold level is set three pixels higher, a stimulation cycle is initiated, the Stimulation flag is set, and the routine is exited. While in the EMG level monitoring (“Ready”) mode, the threshold is checked for upward adjustment every ½ second, ad infinitum.

The devices and methods claimed herein can be made and executed without undue experimentation in light of the present disclosure. While the devices and methods of this invention have been described in terms of particular embodiments, it will be apparent to those of skill in the art that variations may be applied to the systems and methods and/or in the steps or in the sequence of steps of the methods described herein without departing from the concept, spirit and scope of the invention. More specifically, it will be apparent that certain components may be substituted for the components described herein while the same or similar results would be achieved. All such similar substitutes and modifications apparent to those skilled in the art are deemed to be within the spirit, scope and concept of the invention as defined by the appended claims.

SPECIFICATIONS

| EMG Sensitivity: | | |
|---|---|---|
| Stroke rehabilitation mode | 1.25-1000 | μV |
| Spinal cord injury mode | 0.25-25 | μV (more sensitive) |
| | 0.50-50 | μV (less sensitive) |
| Sample Rate: | 2500-3300 | samples/sec |
| Noise Suppression: | 113-120 | dB at 100 Hz |
| Output Current: | 0-100 | mA into 1 Kohm |
| Pulse Width: | 50-400 | μsec |
| Frequency: | 2-160 | Hz |
| Time On: | 0.5-30 | sec |
| Time Off: | 0.1-60 | sec |
| Ramp-up and down: | 0.1-6 | sec |
| Waveform: | Biphasic | |
| Size: | 7.9 × 6.8 × 3.0 | inches |
| Weight: | 22 | oz. |
| Accessories: | 3 | surface sensors |

All of the COMPOSITIONS, METHODS and APPARATUS disclosed and claimed herein can be made and executed without undue experimentation in light of the present disclosure. While the methods and apparatus of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain related components may be substituted for the components described herein while the same or similar results would be achieved. All such similar substitutes and modifications apparent to those skilled in the art are deemed to be within the spirit, scope and concept of the invention as defined by the appended claims.

1. A method of using electrical signals originating in an individual's brain and terminating at an impaired muscle to rehabilitate individuals with motor deficiencies comprising:
(a) prompting the individual to attempt to contract an impaired muscle;
(b) detecting an electrical signal within the impaired muscle using electrodes placed on the individual's skin near the impaired muscle;
(c) transmitting the electrical signal to a microprocessor;
(d) checking the pattern of the electrical signal against a mathematical algorithm;
(e) determining whether or not an attempt to move the impaired muscle has been made;
(f) measuring the strength of the electrical signals; and
(g) sending an electric current to an electrode in contact with the individual's skin to cause a muscle contraction if the strength of the electrical signal is larger than a first threshold value.

2. The method of claim 1 further comprising the step of displaying the strength of the electrical signal on a visual display.

3. The method of claim 1 further comprising the step of setting a second threshold value higher than the first threshold value if the first threshold value is reached in a prior attempt to move the impaired muscle.

4. The method of claim 1 further comprising the step of setting the second threshold value lower than the first threshold value if the first threshold value is not reached in a prior attempt to move the impaired muscle.

5. The method of claim 1 further comprising the step of maintaining the threshold value unchanged.

6. The method of claim 1 further comprising the step of prompting the individual to relax said impaired muscle.

7. The method of claim 1 wherein a prompt is in the form of a sensory cue.

8. The method of claim 7 wherein the prompt is in the form of a visual cue.

9. The method of claim 7 wherein the prompt is in the form of an auditory cue.

10. The method of claim 1 further comprising the step of recording the data received and transmitted by said microprocessor.

11. The method of claim 1 further comprising the step of reducing electrical noise by incorporating a floating, amplified grounding device.

12. A muscular therapy device comprising:
(a) at least two sensors for detecting electrical signals within a muscle;
(b) said sensors in physical contact with a portion of skin near the muscle;
(c) said sensors in electrical contact with a microprocessor;
(d) said microprocessor capable of deciphering from a pattern of said electrical signals whether or not an attempt to move said muscle has been made;
(e) said microprocessor capable of communicating with a display device;
(f) said microprocessor capable of setting a threshold value after every attempt to move the muscle;
(g) said threshold value used to determine when the strength of said attempt is sufficient to warrant a reward; and
(h) said reward in the form of an electrical current sent from said microprocessor to said sensors for causing a visible muscle contraction.

13. The muscular therapy device of claim 10 wherein said microprocessor produces an auditory cue.

14. The muscular therapy device of claim 10 further comprising a memory means for storing information obtained by said microprocessor.

15. The muscular therapy device of claim 10 wherein the sensors cover an area of skin measuring about 1 to 4 square inches.

16. The muscular therapy device of claim 10 wherein the microprocessor is capable of communicating with a display device.

17. The muscular therapy device of claim 14 wherein the display device is angled toward a user when the user is in a reclined position.

18. The muscular therapy device of claim 10 wherein the sensors can detect electrical impulses of about 0.2 to about 2000 μV.

19. The muscular therapy device of claim 10 wherein the microprocessor is capable of analyzing at least 3,000 signals per second.

20. The muscular therapy device of claim 10 further comprising a floating, amplified grounding device for reducing electrical noise in an EMG input of the device.

21. An improvement to a muscular therapy device, the improvement comprising:
(a) means for detecting electrical impulses of about 0.2 to about 2000 μV; and
(b) means for sampling an electrical signal at least 3000 times per second.

22. A method of using electrical signals originating in an individual's brain and terminating at an impaired muscle to rehabilitate individuals with motor deficiencies comprising:
(a) prompting the individual to attempt to contract an impaired muscle;
(b) detecting an electrical signal within the impaired muscle using electrodes placed on the individual's skin near the impaired muscle;
(c) transmitting the electrical signal to a microprocessor;
(d) checking the pattern of the electrical signal against a mathematical algorithm;
(e) determining whether or not an attempt to move the impaired muscle has been made;
(f) measuring the strength of the electrical signals;
(g) sending an electric current to an electrode in contact with the individual's skin to cause a muscle contraction if the strength of the electrical signal is larger than a first threshold value;
(h) detecting electrical impulses of about 0.2 to about 2000 μV;
(i) analyzing at least 3,000 signals per second; and
(j) utilizing a floating, amplified grounding device for reducing electrical noise.

23. A muscular therapy device comprising:
(a) at least two sensors for detecting electrical signals within a muscle;
(b) said sensors in physical contact with a portion of skin near the muscle;
(c) said sensors in electrical contact with a microprocessor;
(d) said microprocessor capable of deciphering from a pattern of said electrical signals whether or not an attempt to move said muscle has been made;
(e) said microprocessor capable of communicating with a display device;
(f) said microprocessor capable of setting a threshold value after every attempt to move the muscle;
(g) said threshold value used to determine when the strength of said attempt is sufficient to warrant a reward;
(h) said reward in the form of an electrical current sent from said microprocessor to said sensors for causing a visible muscle contraction;
(i) said sensors detecting electrical impulses of about 0.2 to about 2000 μV;
(j) said microprocessor capable of analyzing at least 3,000 signals per second; and
(k) a floating, amplified grounding device for reducing electrical noise.

24. A method for improving the sensitivity of a muscular therapy device, said improvement comprising:
(a) providing means for detecting electrical impulses of about 0.2 to about 2000 μV; and
(b) providing means for analyzing at least 2,500 signals per second.

25. A method of using electrical signals originating in an individual's brain and terminating at an impaired muscle to rehabilitate individuals with motor deficiencies comprising:
(a) prompting the individual to attempt to contract an impaired muscle;
(b) detecting an electrical signal within the impaired muscle using electrodes placed on the individual's skin near the impaired muscle;
(c) transmitting the electrical signal to a microprocessor;
(d) checking the pattern of the electrical signal against a mathematical algorithm;
(e) determining whether or not an attempt to move the impaired muscle has been made;
(f) measuring the strength of the electrical signals;
(g) sending an electric current to an electrode in contact with the individual's skin to cause a muscle contraction if the strength of the electrical signal is larger than a first threshold value; and
(h) electronically adjusting the threshold value according to a mathematical algorithm.
